(19) United States
(12) Patent Application Publication
(10) Pub. No.: US 2026/0283995 A1
(43) Pub. Date: Sep. 24, 2026

(54) AGENT FOR REGULATING NEUROLOGICAL FUNCTION

(71) Applicant: HONDA MOTOR CO., LTD., MINATO-KU (JP)

(72) Inventors: Toshiaki NAKAI, WAKO-SHI (JP); Aiko MURAI, WAKO-SHI (JP); Rie HANEISHI, WAKO-SHI (JP); Hiroko ISODA, Tsukuba-shi (JP); Farhana FERDOUSI, TSUKUBA-SHI (JP); Kazunori SASAKI, TSUKUBA-SHI (JP); Mitsutoshi NAKAJIMA, TSUKUBA-SHI (JP)

(21) Appl. No.: 19/570,737

(22) Filed: Mar. 18, 2026

(30) Foreign Application Priority Data
Mar. 21, 2025 (JP) 2025--047172
Feb. 25, 2026 (JP) 2026-028468

(51) Int. Cl. A61K 31/22; A61K 31/045; A61K 31/352; A61P 25/22

(52) U.S. Cl. CPC A61K 31/22; A61K 31/045; A61K 31/352; A61P 25/22

(57) ABSTRACT

What is provided is a method for treating olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders, including administering an effective amount of linalyl acetate, α-terpineol or 1,8-cineole to a subject in need thereof.

### BACKGROUND OF THE INVENTION

### Field of the Invention

The present invention relates to an agent for regulating neurological function. Priorities are claimed on Japanese Patent Applications No. 2025-047172, filed on Mar. 21, 2025, and No. 2026-028468, filed on Feb. 25, 2026, the contents of which are incorporated herein by reference.

### Description of Related Art

A wide variety of chemical substances (odor molecules) that are odor components are received by olfactory cells present in the olfactory epithelium within the nasal cavity. Olfactory cells extend olfactory cilia to the surface of the nasal cavity. In the olfactory cilia, a group of intracellular signaling molecules specific to olfactory cells such as olfactory receptors, GTP-binding proteins (Golf), adenylate cyclase III (cAMP synthase), and cAMP-dependent cation channels are accumulated.

Information on odor molecules bound to olfactory receptors is efficiently converted into electrical signals through the continuous action of these signaling molecule groups, leading to neural excitation. This information is sent to the olfactory bulb in the brain, and additionally sent to the higher-order olfactory centers (the piriform cortex, the amygdala, etc.) and the limbic system (the cerebral cortex, the hippocampus, etc.), and odor recognition, discrimination, memory, emotional changes, attraction or avoidance behaviors and the like are induced.

In recent years, the relationship between odor molecules and health functions has been focused upon and has been actively studied. For example, Non-Patent Documents 1 to 6 describe the effects of odor molecules on a living body.

### Non-Patent Documents

[Non-Patent Document 1] Lee S. J., et al., Therapeutic potential of ectopic olfactory and taste receptors, Nat Rev Drug Discov, 18, 116-138, 2019.
[Non-Patent Document 2] Wu J. J., et al., Modulatory effects of aromatherapy massage intervention on electroencephalogram, psychological assessments, salivary cortisol and plasma brain-derived neurotrophic factor, Complement Ther Med, 22(3), 456-462, 2014.
[Non-Patent Document 3] Okuda M., et al., Aromatherapy improves cognitive dysfunction in senescence-accelerated mouse prone 8 by reducing the level of amyloid beta and tau phosphorylation, PLoS One, 15(10), e0240378, 2020.
[Non-Patent Document 4] Xu J., et al., The Anti-depressant-Like Effect of Vanillin Aroma Involves Serum Magnesium and Brain BDNF, Neuropsychiatry, 8(4), 2018.
[Non-Patent Document 5] Rochefort C., et al., Enriched odor exposure increases the number of newborn neurons in the adult olfactory bulb and improves odor memory, J Neurosci, 22(7), 2679-2689, 2002.
[Non-Patent Document 6] Rusznak Z., et al., Odor Enrichment Increases Hippocampal Neuron Numbers in Mouse, Exp Neurobiol, 27(2), 94-102, 2018.

### SUMMARY OF THE INVENTION

In view of this, an object of the present invention is to provide an agent for regulating neurological function that contains odor molecules as an active component.

The present invention includes the following aspects.
[1] An agent for regulating neurological function that contains linalyl acetate, α-terpineol or 1,8-cineole as an active component.
[2] The agent for regulating neurological function according to [1], which is a therapeutic agent for olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders.
[3] The agent for regulating neurological function according to [1] or [2], which promotes the expression of genes involved in olfactory cilia movement or olfactory ciliary organization in nasal epithelial cells.
[4] The agent for regulating neurological function according to [3], wherein the genes involved in olfactory cilia movement or olfactory ciliary organization include genes associated with the gene ontology terms cilium movement, cilium movement involved in cell motility, cilium or flagellum-dependent cell motility, cilium-dependent cell motility, and cilium organization.
[5] The agent for regulating neurological function according to any one of [1] to [4], which promotes the expression of genes involved in lipid biosynthesis, lipid metabolism, vitamin metabolism or carbohydrate metabolism in the hippocampus.
[6] The agent for regulating neurological function according to [5],
   wherein the genes involved in lipid biosynthesis include genes associated with the gene ontology terms lipid biosynthetic process, membrane lipid biosynthetic process, glycerophospholipid biosynthetic process, and sphingolipid biosynthetic process,
   the genes involved in lipid metabolism include genes associated with the gene ontology terms phospholipid metabolic process, membrane lipid metabolic process, sphingolipid metabolic process, ceramide metabolic process, glycerophospholipid metabolic process, phosphatidylinositol metabolic process, regulation of lipid metabolic process, lipid catabolic process, glycolipid metabolic process, fatty acid beta oxidation, and fatty acid oxidation,
   the genes involved in vitamin metabolism include genes associated with the gene ontology term vitamin metabolic process, and
   the genes involved in carbohydrate metabolism include genes associated with the gene ontology terms monocarboxylic acid metabolic process and carbohydrate metabolic process.

The present invention can also include the following aspects.
[P1] An agent for regulating neurological function that contains linalyl acetate, α-terpineol or 1,8-cineole as an active component.
[P2] The agent for regulating neurological function according to [P1], which is a therapeutic agent for olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders.
[P3] The agent for regulating neurological function according to [P1] or [P2], which promotes the expression of genes involved in olfactory cilia movement or olfactory ciliary organization.
[P4] The agent for regulating neurological function according to any one of [P1] to [P3] wherein the genes involved in olfactory cilia movement or olfactory ciliary organization are genes associated with the gene ontology terms cilium movement, cilium movement involved in cell motility, cilium or flagellum-dependent cell motility, cilium-dependent cell motility or cilium organization.

According to the present invention, it is possible to provide an agent for regulating neurological function that contains odor molecules as an active component.

### BRIEF DESCRIPTION OF THE DRAWINGS

FIG. 1 is a graph showing the results of representative quantitative real-time PCR in Experimental Example 1.

FIG. 2 is a graph showing the results of representative quantitative real-time PCR in Experimental Example 2.

FIG. 3 is a graph showing changes in body weight over time and the results of a tail suspension test for mice made to inhale α-terpineol in Experimental Example 3.

FIG. 4 is a graph showing changes in body weight over time and the results of the tail suspension test for mice made to inhale 1,8-cineole in Experimental Example 3.

FIG. 5 is a graph showing changes in body weight over time and the results of the tail suspension test for mice made to inhale linalyl acetate in Experimental Example 3.

FIG. 6 is a graph showing the results of MTT assay in Experimental Example 5.

FIG. 7 is a graph showing the results of gene ontology (GO) enrichment analysis in Experimental Example 7.

FIG. 8 is a graph showing the results of KEGG pathway analysis in Experimental Example 7.

FIG. 9 is a graph showing the results of gene ontology (GO) enrichment analysis in Experimental Example 7.

FIG. 10 is a graph showing the results of KEGG pathway analysis in Experimental Example 7.

### DETAILED DESCRIPTION OF THE INVENTION

### [Notation of Gene Names and Protein Names]

In this specification, human genes and human proteins are written with uppercase letters. In addition, mouse genes are written with the first letter capitalized followed by lowercase letters. In addition, mouse proteins are written with uppercase letters. However, in some cases, human genes, mouse genes, human proteins, and mouse proteins may be written without strict distinction.

### [Agent for Regulating Neurological Function]

In one embodiment, the present invention provides an agent for regulating neurological function that contains linalyl acetate, α-terpineol or 1,8-cineole as an active component.

The inventors attempted to find odor molecules that exhibit activity related to neurological functions such as an arousal effect and improvement in concentration in an in-vehicle space. Then, as will be described below in examples, they conducted screening using an increase in the expression level of a receptor gene for the neurotransmitter dopamine as an index, and found that three types of essential oils (orange blossom water, myrtille essential oil, and petitgrain bigarade essential oil) exhibited an activity to increase the expression level of the dopamine receptor gene.

The inventors further found that α-terpineol, which is a component of orange blossom water, 1,8-cineole, which is a component of myrtille essential oil, and linalyl acetate, which is a component of petitgrain bigarade essential oil, exhibited an activity to increase the expression level of the dopamine receptor gene.

The inventors further found that linalyl acetate, α-terpineol, and 1,8-cineole exhibited anti-depressant/anti-stress effects in vivo through a mouse tail suspension test.

Therefore, the agent for regulating neurological function of the present embodiment can regulate the neurological function of a living body.

In the present embodiment, regulation of neurological functions means, for example, an arousal effect, improvement in concentration and the like. Alternatively, it means treating or improving diseases such as olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue, and anxiety disorders. Therefore, the agent for regulating neurological function of the present embodiment can also be said to be a therapeutic agent for these diseases.

As will be described below in examples, the inventors further found that linalyl acetate specifically induced expression of a group of genes involved in olfactory cilia movement through microarray analysis. In addition, they found that petitgrain bigarade essential oil containing 50% linalyl acetate specifically induced expression of a group of genes involved in olfactory ciliary organization. Therefore, the agent for regulating neurological function of the present embodiment can also be said to be an inducer of expression of genes involved in olfactory cilia movement or olfactory ciliary organization.

When the agent for regulating neurological function of the present embodiment is administered to a living body, the expression of genes involved in olfactory cilia movement or olfactory ciliary organization is induced in nasal epithelial cells, the movement of olfactory cilia having olfactory receptors and the activation of olfactory ciliary organization occur, the functions of olfactory cells are improved, and therefore not only curing of olfactory disorders, but also the effect of improving the brain function by inducing neurotransmitters such as dopamine is expected.

As will be described below in examples, examples of genes involved in olfactory cilia movement or olfactory ciliary organization include genes associated with the gene ontology terms (GO terms) cilium movement, cilium movement involved in cell motility, cilium or flagellum-dependent cell motility, cilium-dependent cell motility, and cilium organization.

Examples of genes associated with the GO term cilium movement include ADCY10, BBOF1, IQCG, CCDC38, SLC9B1, CCDC63, CFAP221, and CFAP100.

Examples of genes associated with the GO term cilium movement involved in cell motility include BBOF1, IQCG, CCDC38, SLC9B1, and CFAP221.

Examples of genes associated with the GO term cilium or flagellum-dependent cell motility include BBOF1, IQCG, CCDC38, SLC9B1, and CFAP221.

Examples of genes associated with the GO term cilium-dependent cell motility include BBOF1, IQCG, CCDC38, SLC9B1, and CFAP221.

Examples of genes associated with the GO term cilium organization include RRP7A, DCDC2, TTC21B, BBOF1, IQCG, CEP19, CCDC38, CCDC63, CFAP221, and CFAP100.

It is thought that, when the expression of these genes is induced, an arousal effect and an effect of improving concentration are obtained. In addition, it is thought that, when the expression of these genes is induced, effects of treating or improving diseases such as olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue, and anxiety disorders are obtained.

As will be described below in examples, when the agent for regulating neurological function of the present embodiment is administered to a living body, the expression of genes involved in lipid biosynthesis, lipid metabolism, vitamin metabolism or carbohydrate metabolism is promoted in the hippocampus.

Examples of genes involved in lipid biosynthesis include genes associated with the gene ontology terms (GO terms) lipid biosynthetic process, membrane lipid biosynthetic process, glycerophospholipid biosynthetic process, and sphingolipid biosynthetic process.

Examples of genes associated with the GO term lipid biosynthetic process include KL, F5, and LBP.

Examples of genes associated with the GO term membrane lipid biosynthetic process include PIGN, HACD3, and HACD4.

Examples of genes associated with the GO term glycerophospholipid biosynthetic process include ABHD4, INPP5F, and PTDSS1.

Examples of genes associated with the GO term sphingolipid biosynthetic process include ELOVL7, HACD3, and HACD4.

Examples of genes involved in lipid metabolism include genes associated with the gene ontology terms phospholipid metabolic process, membrane lipid metabolic process, sphingolipid metabolic process, ceramide metabolic process, glycerophospholipid metabolic process, phosphatidylinositol metabolic process, regulation of lipid metabolic process, lipid catabolic process, glycolipid metabolic process, fatty acid beta oxidation, and fatty acid oxidation.

Examples of genes associated with the GO term phospholipid metabolic process include ENPP2, PLA2G7, and ABHD4.

Examples of genes associated with the GO term membrane lipid metabolic process include ENPP2, GM2A, and PIGN.

Examples of genes associated with the GO term sphingolipid metabolic process include ENPP2, GM2A, and HACD3.

Examples of genes associated with the GO term ceramide metabolic process include GM2A, PLPP2, and PLPP3.

Examples of genes associated with the GO term glycerophospholipid metabolic process include ENPP2, DGKB, and OCRL.

Examples of genes associated with the GO term phosphatidylinositol metabolic process include OCRL, PIGN, and INPP5F.

Examples of genes associated with the GO term regulation of lipid metabolic process include ACAA2, ELOVL7, and MSMO1.

Examples of genes associated with the GO term lipid catabolic process include ENPP2, PLA2G7, and PLA2G5.

Examples of genes associated with the GO term glycolipid metabolic process include PIGN, GM2A, and SLC30A9.

Examples of genes associated with the GO term fatty acid beta oxidation include ACAA2, DECR1, and ETFDH.

Examples of genes associated with the GO term fatty acid oxidation include ECHS1, ECI2, and CROT.

Examples of genes involved in vitamin metabolism include genes associated with the gene ontology term vitamin metabolic process.

Examples of genes associated with the GO term vitamin metabolic process include TTR, ENPP2, and RBP1.

Examples of genes involved in carbohydrate metabolism include genes associated with the gene ontology terms monocarboxylic acid metabolic process and carbohydrate metabolic process.

Examples of genes associated with the GO term monocarboxylic acid metabolic process include CYP39A1, UCP2, and FADS2.

Examples of genes associated with the GO term carbohydrate metabolic process include KL, UCP2, and FOXO1.

It is thought that enhancement of lipid biosynthesis, remodeling, and energy metabolism contributes to maintaining the neuronal membrane integrity and synaptic function in the hippocampus. In addition, it is thought that, by regulating lipid metabolism networks and activating signaling pathways related to cell growth and survival, neuroprotective effects in the hippocampus are exhibited. In addition, it is thought that hippocampus metabolism is more dynamically regulated under stress conditions not only by promoting lipid remodeling and membrane homeostasis but also by enhancing lipid utilization and energy release.

In addition, as will be described below in examples, the agent for regulating neurological function of the present embodiment was administered to a living body, genes with increased expression in the hippocampus were subjected to KEGG pathway analysis, and as a result, pathways such as fatty acid metabolism, fatty acid elongation, biosynthesis of unsaturated fatty acids, sphingolipid signaling, sphingolipid metabolism, cAMP signaling pathway, PI3K-Akt signaling pathway, butanoate metabolism, glycine, serine, and threonine metabolism, and steroid biosynthesis were significantly enriched. These metabolic effects were thought to secure structural resilience and energy balance in the hippocampus under stress-induced conditions.

These results are thought to provide further support that, when the agent for regulating neurological function of the present embodiment is administered to a living body, effects of treating or improving diseases such as olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue, and anxiety disorders are obtained.

In this specification, “containing as an active component” means containing as a main active component, and means containing to an extent that an effect is exhibited. The “effect” herein includes, for example, an effect of increasing the expression level of dopamine receptor genes in nerve cells, an effect of promoting the expression of genes involved in olfactory cilia movement or olfactory ciliary organization in nasal epithelial cells, an arousal effect in vivo, improvement in concentration, and an anti-depressant/anti-stress effect.

In the agent for regulating neurological function of the present embodiment, linalyl acetate, α-terpineol, and 1,8-cineole may be purified compounds. In addition, these purified compounds may be of one type or may be a mixture of two or more types. Alternatively, as long as the effects of the present invention are obtained, a composition containing these compounds and various other components may be used.

For example, the agent for regulating neurological function of the present embodiment may be petitgrain bigarade essential oil containing linalyl acetate as an active component, orange blossom water containing α-terpineol as an active component, or myrtille essential oil containing 1,8-cineole as an active component, or a mixture of two or more thereof.

The agent for regulating neurological function of the present embodiment may be in the form of essential oil or the form of a food composition, or may be formulated as a pharmaceutical composition.

Essential oils can be administered to a living body, for example, by diffusing them with a diffuser and inhaling them through the nose. Alternatively, essential oils may be mixed into a food composition and then ingested by the living body.

The dosage of the essential oil can be appropriately determined depending on the subject's symptoms, body weight, age, sex and the like, and may be administered once a day or about 2 to 4 times a day in a divided manner. For example, when the essential oil is diffused with a diffuser and inhaled through the nose, an adult may inhale air containing an active component (linalyl acetate, α-terpineol or 1,8-cineole) at a concentration of 0.5 to 200 ppm for about 15 minutes to 4 hours per day.

The food composition may be in any form such as a granular form, a particle form, a paste form, a gel form, a solid form or a liquid form. The food composition may appropriately contain various substances known to those skilled in the art that are approved for inclusion in foods, for example, additives such as binders, disintegrants, thickeners, dispersants, reabsorption promoting agents, flavoring agents, buffering agents, surfactants, solubilizing agents, preservatives, emulsifiers, isotonicity agents, stabilizing agents, and pH adjusting agents.

The food composition may be prepared as a health food, a food with function claims, a food for specified health uses, a nutritional supplement food, a food with health functions such as a food with nutrient function, a food for special uses (for example, a food for sick people), a food with disease risk reduction claims, a health supplement food, a supplement or the like.

The supplement may be in the form of, for example, tablets, pills, capsules (including hard capsules, soft capsules, and microcapsules), powders, granules, fine granules, lozenges, liquids (including syrups, emulsions, and suspensions), together with various additives commonly used in production of supplements. In addition, it may be in the form of a semi-liquid or a paste, or a form added to general foods and beverages.

The amount of the food composition ingested can be appropriately determined depending on the subject's symptoms, body weight, age, sex and the like, and may be ingested once a day or about 2 to 4 times a day in a divided manner.

The pharmaceutical composition may contain the above agent for regulating neurological function and a pharmaceutically acceptable carrier. As the pharmaceutically acceptable carrier, those commonly used in formulation of pharmaceutical compositions can be used without particular limitation. For example, the pharmaceutical composition can be administered orally in the form of tablets, granules, powders, capsules or the like, or administered parenterally in the form of injectable preparations, suppositories, topical skin preparations or the like. More specific examples of dosage forms of topical skin preparations include ointments and patches.

When the pharmaceutical composition is in the form of an external preparation, it can be produced by making the active component into a solid, semi-solid or liquid composition. For example, the solid composition can be produced by making the active component alone into a powder form or by adding and mixing an excipient (for example, lactose, mannitol, starch, microcrystalline cellulose, or sucrose), a thickener (for example, natural gums, cellulose derivatives, or acrylic acid polymers) and the like and making the mixture into a powder form. The liquid composition can be produced in the same manner as the injectable preparation. The semi-solid composition may be an aqueous or oily gel agent or a cartilage-like composition. These compositions each may contain a pH regulator (for example, carbonic acid, phosphoric acid, citric acid, hydrochloric acid, or sodium hydroxide), a preservative (for example, parahydroxybenzoates, chlorobutanol, or benzalkonium chloride) and the like. Suppositories can be produced by making the active component into an oily or aqueous solid, semi-solid or liquid composition. Examples of oily bases include glycerides of higher fatty acids [for example, cacao butter, Witepsols (commercially available from Dynamite Nobel AG)], medium-chain fatty acids[for example, Miglyols (commercially available from Dynamite Nobel AG)], and vegetable oils (for example, sesame oil, soybean oil, and cottonseed oil). Examples of aqueous bases include polyethylene glycols and propylene glycol. In addition, examples of aqueous gel bases include natural gums, cellulose derivatives, vinyl polymers, and acrylic acid polymers.

When the pharmaceutical composition is in the form of an oral preparation, it can be produced by adding an excipient (for example, lactose, sucrose, starch, or mannitol), a disintegrant (for example, calcium carbonate, or calcium carboxymethylcellulose), a binder (for example, pregelatinized starch, gum arabic, carboxymethylcellulose, polyvinylpyrrolidone, or hydroxypropylcellulose) or a lubricant (for example, talc, magnesium stearate, or polyethylene glycol 6000) to an active component, compressing and molding the mixture, and then, as necessary, performing coating by a method known in the art for the purpose of taste masking, enteric properties or sustained release. As the coating agent, for example, ethyl cellulose, hydroxymethyl cellulose, polyoxyethylene glycol, cellulose acetate phthalate, hydroxypropyl methylcellulose phthalate, Eudragit (registered trademark, commercially available from Rohm GmbH & Co. KG., Germany, methacrylic acid-acrylic acid copolymer) and the like can be used.

When the pharmaceutical composition is in the form of an injectable preparation, it can be produced by dissolving, suspending or emulsifying the active component in an aqueous solvent (for example, distilled water, physiological saline, Ringer's solution, etc.) or an oily solvent (for example, vegetable oils such as olive oil, sesame oil, cottonseed oil, and corn oil, propylene glycol, etc.) together with a dispersant (for example, Tween80 (registered trademark, commercially available from Atlas Powder Company, USA), HCO60 (registered trademark, commercially available from Nikko Chemicals Co., Ltd.), polyethylene glycol, carboxymethylcellulose, sodium alginate, etc.), a preservative (for example, methylparaben, propylparaben, benzyl alcohol, chlorobutanol, phenol, etc.), and an isotonicity agent (for example, sodium chloride, glycerin, sorbitol, glucose, invert sugar, etc.). In this case, additives such as solubilizing agents (for example, sodium salicylate, sodium acetate, etc.), stabilizing agents (for example, human serum albumin, etc.), and soothing agents (for example, benzalkonium chloride, procaine hydrochloride, etc.) may be added as desired.

The dosage of the pharmaceutical composition can be appropriately determined depending on the subject's symptoms, body weight, age, sex and the like, and may be administered once a day or about 2 to 4 times a day in a divided manner.

When the essential oil, the food composition or the pharmaceutical composition is administered to a living body, the expression of olfactory cilia movement and tissue-related genes is promoted, and effects of preventing, alleviating or treating symptoms or diseases mediated by olfactory cilia movement and tissue-related genes are obtained. Examples of diseases include those described above.

### Other Embodiments

In one embodiment, the present invention provides a method for treating olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders, including administering an effective amount of linalyl acetate, α-terpineol or 1,8-cineole to a subject in need thereof.

In one embodiment, the present invention provides a use of linalyl acetate, α-terpineol or 1,8-cineole for producing a therapeutic agent for olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders.

In these embodiments, linalyl acetate, α-terpineol, 1,8-cineole, administration methods thereof and dosages thereof are same as those described above.

### EXAMPLES

The present invention will be described below in more detail with reference to examples, but the present invention is not limited to the following example.

### Experimental Example 1

(Searching for Essential Oils that Increase Expression of Dopamine Receptor Genes)

SH-SY5Y cells, which are a human neuroblastoma cell line, were seeded on a 10 cm² dish at 3.7×10⁶ cells/dish, and cultured for 24 hours. After 24 hours, the medium was replaced with a medium supplemented with various essential oils, and the cells were additionally incubated for 24 hours.

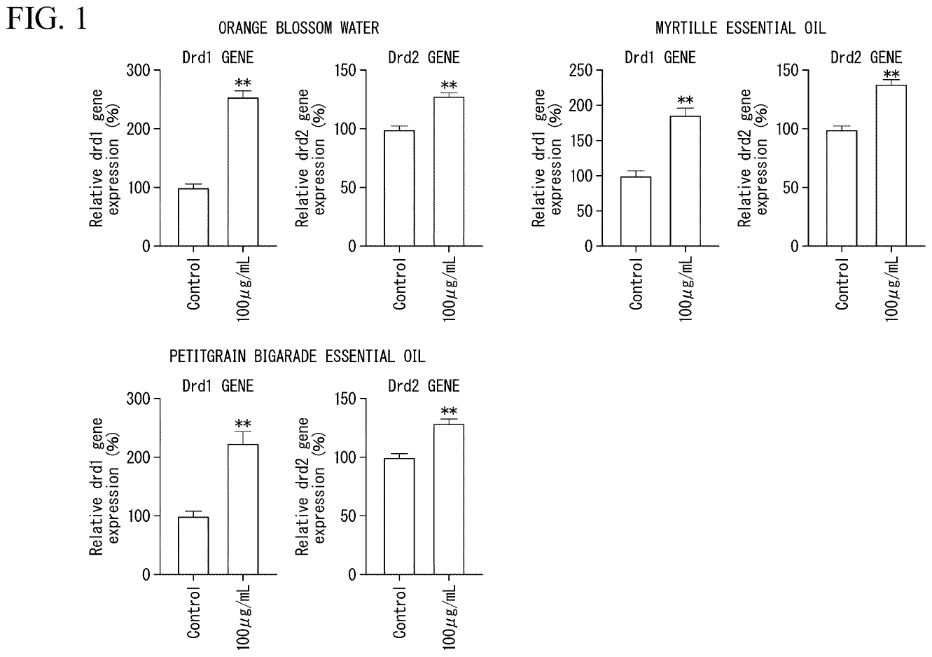

Subsequently, RNA was purified using ISOGEN (Nippon Gene), and the expression levels of the Dopamine receptor D1 (Drd1) gene and the Dopamine receptor D2 (Drd2) gene were quantified by quantitative real-time PCR. FIG. 1 is a graph showing the results of representative quantitative real-time PCR. In FIG. 1, “**” indicates a significant difference at p<0.01. In addition, “Control” indicates a control to which no essential oil was added.

As a result, it was found that orange blossom water, myrtille essential oil, and petitgrain bigarade essential oil, which are essential oils, each significantly improved the expression levels of the Drd1 gene and the Drd2 gene at a concentration of 100 g/mL.

### Experimental Example 2

(Identification of Components that Increase Expression of Dopamine Receptor Gene)

Components in orange blossom water, myrtille essential oil, and petitgrain bigarade essential oil, which improved the expression levels of the Drd1 gene and the Drd2 gene, were identified.

SH-SY5Y cells, which are a human neuroblastoma cell line, were seeded on a 10 cm² dish at 3.7×10⁶ cells/dish, and the cells were cultured for 24 hours. After 24 hours, the medium was replaced with a medium supplemented with various candidate components, and the cells were additionally incubated for 24 hours.

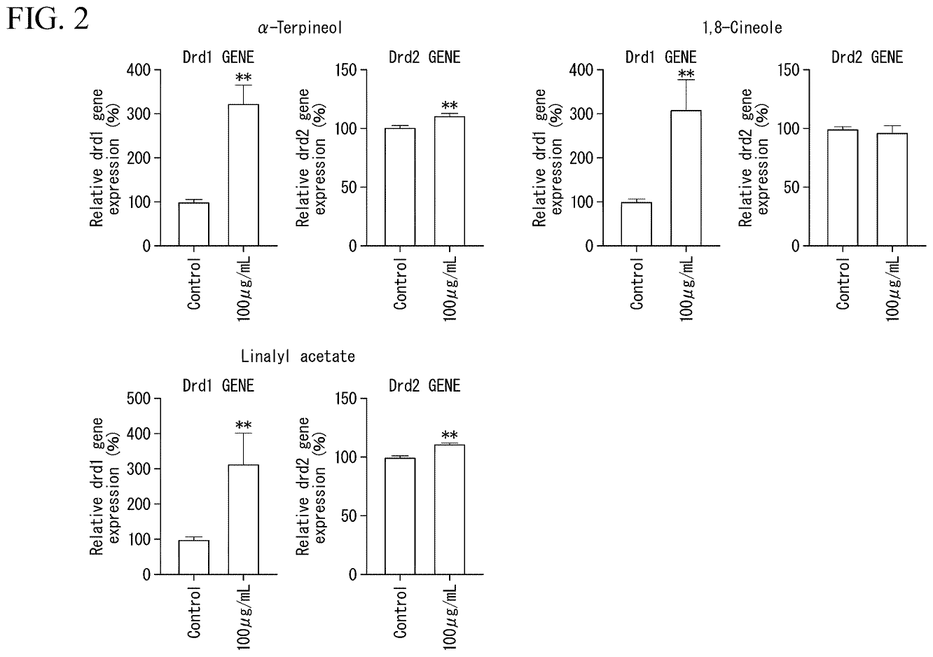

Subsequently, RNA was purified using ISOGEN (Nippon Gene), and the expression levels of the Drd1 gene and the Drd2 gene were quantified by quantitative real-time PCR. FIG. 2 is a graph showing the results of representative quantitative real-time PCR. In FIG. 2, “**” indicates a significant difference at p<0.01. In addition, “Control” indicates a control to which no candidate component was added.

As a result, it was found that α-terpineol, which is a component of orange blossom water, 1,8-cineole, which is a component of myrtille essential oil, and linalyl acetate, which is a component of petitgrain bigarade essential oil, each improved the expression levels of the Drd1 gene and the Drd2 gene at a concentration of 100 g/mL. The chemical formulae of α-terpineol, 1,8-cineole, and linalyl acetate are shown below.

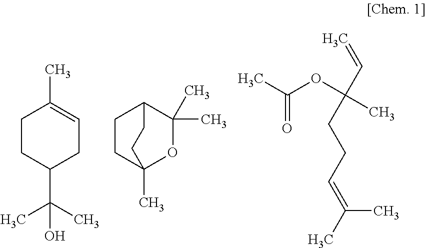

α-terpineol1,8-cineolelinalyl acetate

### Experimental Example 3

### (Evaluation of Anti-Stress Effect by In Vivo Experiment)

The effects of α-terpineol, 1,8-cineole, and linalyl acetate on depressive behavior in vivo were examined by a tail suspension test using mice. The tail suspension test (TST) is a test that measures the time during which a mouse hanged upside down struggles and is used as an index of depressive-like behavior. The hanged mouse moves around trying to escape, but the time during which it does not move (immobility time) gradually increases. It is known that mice administered with an antidepressant showed a decrease in the immobility time in the tail suspension test. It is interpreted that a longer immobility time indicates an increase in depressive-like behavior, and a shorter immobility time indicates a decrease in depressive-like behavior (relief of stress, improvement of motivation, etc.).

Mice (ICR mice, male, 8-week old) were individually reared for 7 days and acclimated. Subsequently, the mice were put into a chamber with an aroma diffuser (product name “Nano Nebula”) installed, and were made to inhale samples to be described below for 30 minutes per day for 7 days. In addition, after inhaling the samples and following a 1-hour interval, the mice were subjected to the tail suspension test every day for 7 days. In addition, the body weight was measured every day.

The samples used were 1% and 10% α-terpineol, 1% and 10% 1,8-cineole, 1% and 10% linalyl acetate, and a control (only a solvent, 1% Tween80).

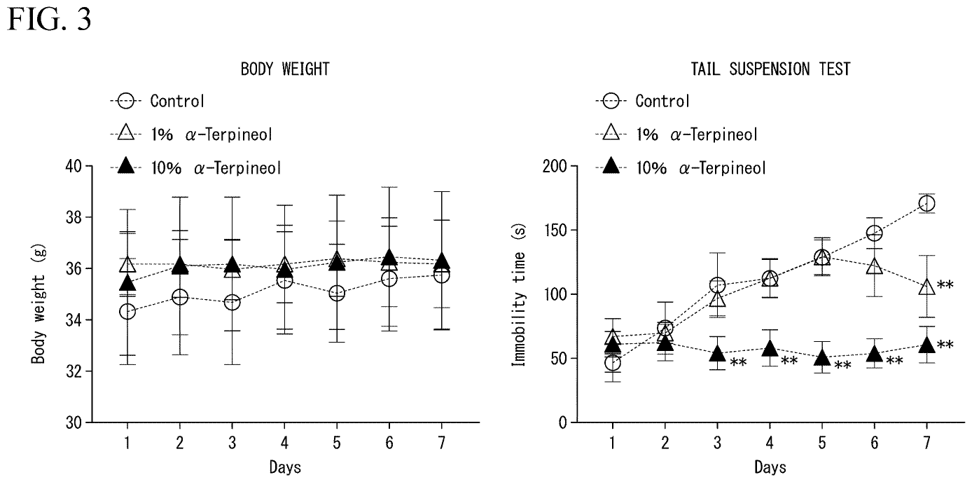

FIG. 3 is a graph showing changes in body weight over time and the results of the tail suspension test for mice made to inhale α-terpineol. In FIG. 3, “**” indicates a significant difference at p<0.01.

As a result, no significant changes in body weight were observed during the test period, and it was thought that α-terpineol did not exhibit severe toxicity. In addition, in the 10% α-terpineol inhalation group, a significant decrease in immobility time was observed from day 3 onward. In addition, in the 10% α-terpineol inhalation group, compared to the 1% α-terpineol inhalation group, a greater effect of decreasing the immobility time was observed.

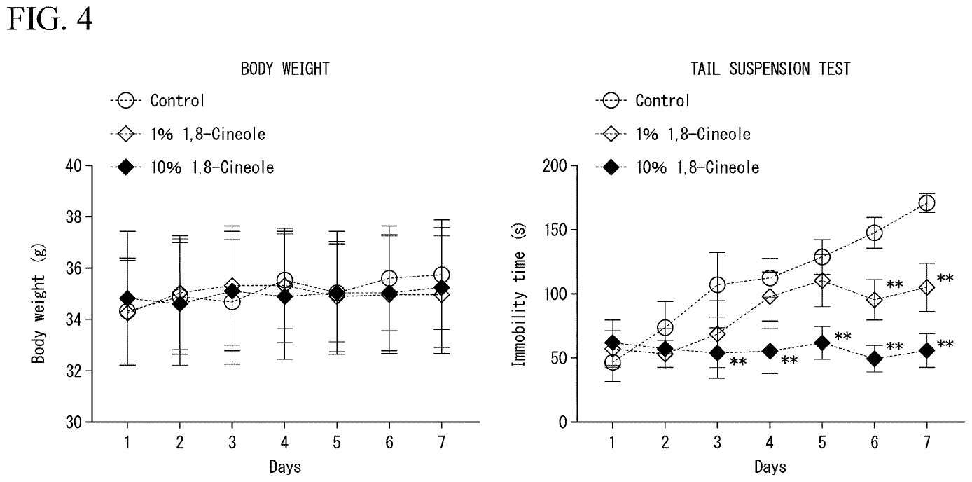

FIG. 4 is a graph showing changes in body weight over time and the results of the tail suspension test for mice made to inhale 1,8-cineole. In FIG. 4, “**” indicates a significant difference at p<0.01.

As a result, no significant changes in body weight were observed during the test period, and it was thought that 1,8-cineole did not exhibit severe toxicity. In addition, in the 10% 1,8-cineole inhalation group, a significant decrease in immobility time was observed from day 3 onward. In addition, in the 10% 1,8-cineole inhalation group, compared to the 1% 1,8-cineole inhalation group, a greater effect of decreasing the immobility time was observed.

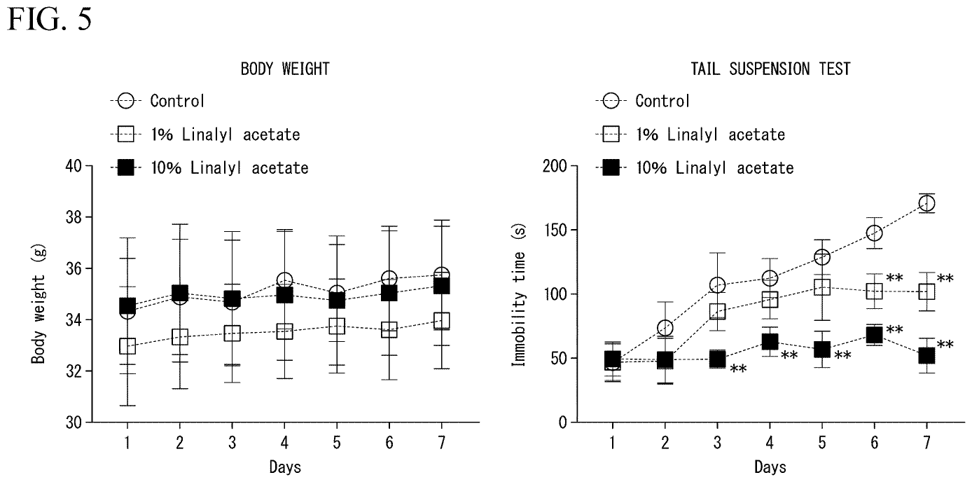

FIG. 5 is a graph showing changes in body weight over time and the results of the tail suspension test for mice made to inhale linalyl acetate. In FIG. 5, “**” indicates a significant difference at p<0.01. As a result, no significant changes in body weight were observed during the test period, and it was thought that linalyl acetate did not exhibit severe toxicity. In addition, in the 10% linalyl acetate inhalation group, a significant decrease in immobility time was observed from day 3 onward. In addition, in the 10% linalyl acetate inhalation group, compared to the 1% linalyl acetate inhalation group, a greater effect of decreasing the immobility time was observed.

### Experimental Example 4

### (Measurement of Gas Concentration in Chamber)

The concentrations of α-terpineol, 1,8-cineole, and linalyl acetate in the chamber used in Experimental Example 3 were measured.

The following Table 1 shows the results of calculating the concentrations of the components in the chamber after mists of 10% α-terpineol, 10% 1,8-cineole, and 10% linalyl acetate were generated for 30 minutes using an aroma diffuser (product name “Nano Nebula,” commercially available from Entrex. Inc.). The following Table 1 also shows the vapor pressure and the gas-liquid equilibrium concentration of the components. The gas-liquid equilibrium concentration is a volumetric value.

TABLE 1

| | Vapor | Gas-liquid | Concentration in |
|---|---|---|---|
| | pressure | equilibrium | chamber |
| | (mmHg) | concentration (ppm) | (ppm) |
| α-Terpineol | 0.028 | 36.8 | 24.2 |
| 1,8-Cineole | 1.90 | 2500 | 232 |
| Linalyl acetate | 0.116 | 153 | 41.1 |

As a result, it was found that, after 30 minutes of mist generation, the mist concentration was lower than the gas-liquid equilibrium concentration. Although the mist was made up of fine droplets, its surface area became enormous, and thus it was estimated that it evaporated relatively quickly to become an essential oil gas at several tensof ppm to several hundreds of ppm.

### Experimental Example 5

(Evaluation of Toxicity of Essential Oils and their Components on Human Nasal Epithelial Cells)

Human nasal epithelial cells were obtained from PromoCell. The human nasal epithelial cells were seeded on a 96-well plate at 2.0×10⁴ cells/well, and cultured for 24 hours. After 24 hours, the medium was replaced with a medium supplemented with essential oils or their components, and the cells were additionally incubated for 24 hours.

The essential oils used were orange blossom water, myrtille essential oil, and petitgrain bigarade essential oil at concentrations of 1,5,10,25,50, and 100 g/mL. The essential oil components used were 1,5,10,25,50, and 100 Mα-terpineol, 1,8-cineole, and linalyl acetate.

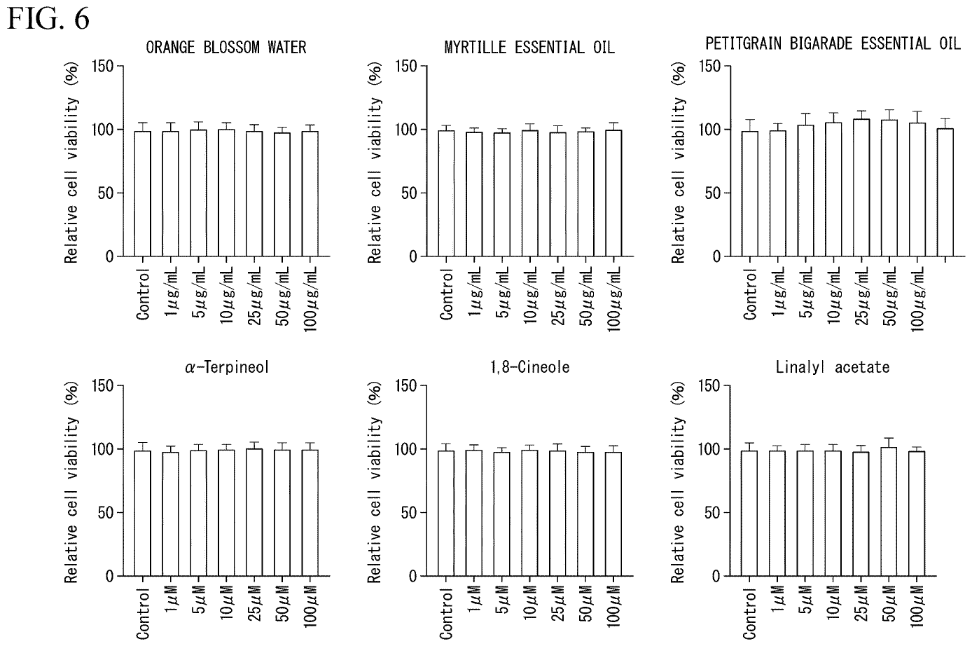

Subsequently, the cell viability of the human nasal epithelial cells was measured through MTT assay. FIG. 6 is a graph showing the results of MTT assay. As a result, none of the essential oils or their components exhibited significant cytotoxicity at any concentration.

### Experimental Example 6

### (Microarray Analysis of Human Nasal Epithelial Cells)

Human nasal epithelial cells were incubated in a medium supplemented with essential oils or their components, and changes in gene expression were analyzed through microarray analysis.

First, human nasal epithelial cells were seeded on a 6-well plate at 6.0×10¹ cells/well, and cultured for 24 hours. After 24 hours, the medium was replaced with a medium supplemented with essential oils or their components, and the cells were additionally incubated for 24 hours.

The essential oils used were orange blossom water, myrtille essential oil, and petitgrain bigarade essential oil at a concentration of 100 g/mL. The essential oil components used were 100 Mα-terpineol, 1,8-cineole, and linalyl acetate.

Subsequently, RNA was extracted using ISOGEN (Nippon Gene), and microarray analysis was performed using Clariom S Assay (Applied Biosystems). 20,000 or more genes were analyzed. Data was analyzed using software such as Database for Annotation and Visualization and Integrated Discovery (DAVID).

The following Table 2 shows the genes with increased expression in human nasal epithelial cells supplemented with orange blossom water, olfactory receptor genes with increased expression, and their functions. As a result of gene ontology (GO) enrichment analysis, in the human nasal epithelial cells supplemented with orange blossom water, G protein-coupled receptor signaling pathway, calcium ion transport, transmembrane transport, receptor activity, and synaptic function were controlled. In addition, in KEGG pathway analysis, synapse-related functions were significant.

The following Table 3 shows the genes with increased expression in human nasal epithelial cells supplemented with myrtille essential oil, olfactory receptor genes with increased expression, and their functions. As a result of gene ontology (GO) enrichment analysis, in the human nasal epithelial cells supplemented with myrtille essential oil, most biological events of olfactory signaling were controlled. In addition, activation of olfactory receptors in the nasal epithelium was confirmed. In addition, transmembrane transport, sensory organ development, olfactory sensory perception, receptor activity, and synaptic functions of both development and transmission were controlled. In addition, in KEGG pathway analysis, signaling and neuroactive ligand-receptor interaction regulation were clearly indicated. The neuroactive ligand-receptor interaction pathway was mainly composed of a group of neuroreceptor genes such as dopamine receptors.

The following Table 4 shows the genes with increased expression in human nasal epithelial cells supplemented with petitgrain bigarade essential oil, olfactory receptor genes with increased expression, and their functions. As a result of gene ontology (GO) enrichment analysis, in the human nasal epithelial cells supplemented with petitgrain bigarade essential oil, pathways related to olfactory signaling were weakly regulated, but sensory organ development, sensory perception, cilia organization, and synaptic function were significantly controlled. In addition, in KEGG pathway analysis, neurotrophic factor signaling pathway (related to development), neuroactive ligand-receptor interaction (related to synapses and neurotransmitters), and Toll-like receptors (related to nasal inflammation, potential anti-inflammatory effects) were detected.

The following Table 5 shows the genes with increased expression in human nasal epithelial cells supplemented with α-terpineol, olfactory receptor genes with increased expression, and their functions. As a result of gene ontology (GO) enrichment analysis, in the human nasal epithelial cells supplemented with α-terpineol, both the G protein signaling pathway and sensory function were controlled, but the transmembrane transport activity and synaptic function were weakly regulated. In KEGG pathway analysis, it was clearly indicated that olfactory transduction was specifically regulated. α-terpineol was considered to act on the early events of olfactory signaling, and to have little activity in membrane transport.

The following Table 6 shows the genes with increased expression in human nasal epithelial cells supplemented with 1,8-cineole, olfactory receptor genes with increased expression, and their functions. As a result of gene ontology (GO) enrichment analysis, in the human nasal epithelial cells supplemented with 1,8-cineole, all three major biological events of olfactory signaling were controlled. In KEGG pathway analysis, the greatest effect was observed in synaptic function.

The following Table 7 shows the genes with increased expression in human nasal epithelial cells supplemented with linalyl acetate, olfactory receptor genes with increased expression, and their functions. As a result of gene ontology (GO) enrichment analysis, linalyl acetate significantly controlled the G protein signaling pathway. Among all samples, linalyl acetate exhibited the greatest effects on cilia and cell motility. In the gene ontology terms, cilium movement, cilium movement involved in cell motility, cilium or flagellum-dependent cell motility, cilium-dependent cell motility, cilium organization and the like were enriched. Interestingly, biological processes related to synaptic function were not significant, but in KEGG pathway analysis, linalyl acetate exhibited a significant effect on dopaminergic synapses.

TABLE 2
Orange blossom water

| Gene | | Fold | | |
|---|---|---|---|---|
| Symbol | Description | Change | P-val | Function |
| PDE4B | phosphodiesterase 4B, | 2.11 | 0.0036 | calcium channel regulating |
| | CAMP-specific | | | activity, cAMP binding, |
| | | | | transmembrane transporter |
| | | | | binding, cAMP-mediated |
| | | | | signaling, and regulation of |
| | | | | calcium ion transmembrane |
| | | | | transport via |
| | | | | high-voltage-gated calcium |
| | | | | channel |
| F7 | coagulation factor VII | 2.07 | 0.001 | signal receptor binding and |
| | | | | calcium ion binding |
| CD5 | CD5 molecule | 2.03 | 0.0166 | signal receptor activity |
| CTPS2 | CTP synthase 2 | 1.91 | 0.0113 | ATP binding |
| OR52E1 | olfactory receptor, family 52, | 1.9 | 0.0039 | G protein-coupled receptor |
| | subfamily E, member 1 | | | activity, olfactory receptor |
| OR7D4 | olfactory receptor, family 7, | 1.65 | 0.0115 | activity, and detection of |
| | subfamily D, member 4 | | | chemical stimuli involved in |
| OR10K2 | olfactory receptor, family 10, | 1.62 | 0.0015 | olfactory sensory perception |
| | subfamily K, member 2 | | | |
| OR13C4 | olfactory receptor, family 13, | 1.49 | 0.0272 | |
| | subfamily C, member 4 | | | |
| OR4E2 | olfactory receptor, family 4, | 1.48 | 0.0376 | |
| | subfamily E, member 2 | | | |
| OR6J1 | olfactory receptor, family 6, | 1.45 | 0.0037 | |
| | subfamily J, member 1 | | | |
| OR52W1 | olfactory receptor, family 52, | 1.44 | 0.0428 | |
| | subfamily W, member 1 | | | |
| OR5AC2 | olfactory receptor, family 5, | 1.33 | 0.0297 | |
| | subfamily AC, member 2 | | | |
| OR4K3 | olfactory receptor, family 4, | 1.27 | 0.0453 | |
| | subfamily K, member 3 | | | |

TABLE 3
Myrtille essential oil

| Gene | | Fold | | |
|---|---|---|---|---|
| Symbol | Description | Change | P-val | Function |
| NQO1 | NAD(P)H dehydrogenase, | 2.71 | 2.14E−06 | synaptic transmission, |
| | quinone 1 | | | cholinergic |
| OR5V1 | olfactory receptor, family | 2.15 | 0.0001 | G protein-coupled |
| | 5, subfamily V, member 1 | | | receptor activity, |
| OR8J1 | olfactory receptor, family | 1.95 | 0.0007 | olfactory receptor |
| | 8, subfamily J, member 1 | | | activity, and detection |
| OR51B5 | olfactory receptor, family | 1.8 | 0.0418 | of chemical stimuli |
| | 51, subfamily B, member 5 | | | involved in olfactory |
| OBP2A | odorant binding protein 2A | 1.78 | 0.0019 | sensory perception. A |
| OR4D2 | olfactory receptor, family | 1.71 | 0.0233 | total of 65 |
| | 4, subfamily D, member 2 | | | olfactory-related |
| OR10V1 | olfactory receptor, family | 1.68 | 0.0378 | differentially expressed |
| | 10, subfamily V, member 1 | | | genes were detected. |
| OR5212 | olfactory receptor, family | 1.68 | 0.0054 | |
| | 52, subfamily I, member 2 | | | |
| OR6A2 | olfactory receptor, family | 1.67 | 0.0068 | |
| | 6, subfamily A, member 2 | | | |
| OR4C3 | olfactory receptor, family | 1.66 | 0.04 | |
| | 4, subfamily C, member 3 | | | |
| OR51J1 | olfactory receptor, family | 1.64 | 0.0006 | |
| | 51, subfamily J, member 1 | | | |

TABLE 4
Petitgrain bigarade essential oil

| Gene | | Fold | | |
|---|---|---|---|---|
| Symbol | Description | Change | P-val | Function |
| OR2M3 | olfactory receptor, family 2, | 2.17 | 0.0015 | G protein-coupled |
| | subfamily M, member 3 | | | receptor activity, |
| OR1L8 | olfactory receptor, family 1, | 1.92 | 0.0046 | olfactory receptor |
| | subfamily L, member 8 | | | activity, and detection |
| OR2M4 | olfactory receptor, family 2, | 1.69 | 0.0051 | of chemical stimuli |
| | subfamily M, member 4 | | | involved in olfactory |
| OR5C1 | olfactory receptor, family 5, | 1.62 | 0.0384 | sensory perception |
| | subfamily C, member 1 | | | |
| OR4D9 | olfactory receptor, family 4, | 1.59 | 0.0008 | |
| | subfamily D, member 9 | | | |
| OR10W1 | olfactory receptor, family 10, | 1.57 | 0.0053 | |
| | subfamily W, member 1 | | | |
| OR51B4 | olfactory receptor, family 51, | 1.55 | 0.0221 | |
| | subfamily B, member 4 | | | |
| OR13H1 | olfactory receptor, family 13, | 1.52 | 0.0111 | |
| | subfamily H, member 1 | | | |
| OR4Q2 | olfactory receptor, family 4, | 1.5 | 0.0435 | |
| | subfamily Q, member 2 | | | |
| | (gene/pseudogene) | | | |
| OR1A1 | olfactory receptor, family 1, | 1.49 | 0.0276 | |
| | subfamily A, member 1 | | | |
| OR5K2 | olfactory receptor, family 5, | 1.48 | 0.03 | |
| | subfamily K, member 2 | | | |

TABLE 5
α-Terpineol

| Gene | | Fold | | |
|---|---|---|---|---|
| Symbol | Description | Change | P-val | Function |
| OR5D18 | olfactory receptor, family 5, | 1.79 | 0.003 | G protein-coupled |
| | subfamily D, member 18 | | | receptor activity, |
| OLFML1 | olfactomedin like 1 | 1.67 | 0.0228 | olfactory receptor |
| OCM2 | oncomodulin 2 | 1.65 | 0.0132 | activity, and detection |
| OR13C4 | olfactory receptor, family | 1.63 | 0.0072 | of chemical stimuli |
| | 13, subfamily C, member 4 | | | involved in olfactory |
| OR10S1 | olfactory receptor, family | 1.58 | 0.026 | sensory perception |
| | 10, subfamily S, member 1 | | | |
| OR2K2 | olfactory receptor, family 2, | 1.53 | 0.0438 | |
| | subfamily K, member 2 | | | |
| ORMDL2 | ORMDL sphingolipid | 1.52 | 0.0012 | |
| | biosynthesis regulator 2 | | | |
| OR6A2 | olfactory receptor, family 6, | 1.42 | 0.0496 | |
| | subfamily A, member 2 | | | |
| OR13C3 | olfactory receptor, family | 1.39 | 0.016 | |
| | 13, subfamily C, member 3 | | | |
| OCM | oncomodulin | 1.39 | 0.0425 | |
| ORM1 | orosomucoid1 | 1.39 | 0.0354 | |

TABLE 6
1,8-Cineole

| Gene | | Fold | | |
|---|---|---|---|---|
| Symbol | Description | Change | P-val | Function |
| OR56A1 | olfactory receptor, family | 1.77 | 0.0059 | G protein-coupled |
| | 56, subfamily A, member 1 | | | receptor activity, |
| OR7D4 | olfactory receptor, family | 1.7 | 0.008 | olfactory receptor |
| | 7, subfamily D, member 4 | | | activity, and detection |
| OR6C74 | olfactory receptor, family | 1.61 | 0.0101 | of chemical stimuli |
| | 6, subfamily C, member 74 | | | involved in olfactory |
| OR2M4 | olfactory receptor, family | 1.57 | 0.0142 | sensory perception |
| | 2, subfamily M, member 4 | | | |
| OR5212 | olfactory receptor, family | 1.55 | 0.0149 | |
| | 52, subfamily I, member 2 | | | |
| OR2T29 | olfactory receptor, family | 1.51 | 0.0201 | |
| | 2, subfamily T, member 29 | | | |
| OLAH | oleoyl-ACP hydrolase | 1.5 | 0.0397 | |
| OBP2B | odorant binding protein 2B | 1.49 | 0.0202 | |
| OR2Z1 | olfactory receptor, family | 1.42 | 0.0111 | |
| | 2, subfamily Z, member 1 | | | |
| OR1C1 | olfactory receptor, family | 1.38 | 0.0222 | |
| | 1, subfamily C, member 1 | | | |
| OR5B3 | olfactory receptor, family | 1.37 | 0.0478 | |
| | 5, subfamily B, member 3 | | | |
| OBP2B | odorant binding protein 2B | 1.37 | 0.0438 | |
| OBP2B | odorant binding protein 2B | 1.35 | 0.0122 | |

TABLE 7
Linalyl acetate

| Gene | | Fold | | |
|---|---|---|---|---|
| Symbol | Description | Change | P-val | Function |
| OR6A2 | olfactory receptor, family 6, | 2.06 | 0.0006 | G protein-coupled |
| | subfamily A, member 2 | | | receptor activity, |
| OR51A4 | olfactory receptor, family | 1.93 | 0.0032 | olfactory receptor |
| | 51, subfamily A, member 4 | | | activity, and detection |
| OR7A5 | olfactory receptor, family 7, | 1.92 | 0.0028 | of chemical stimuli |
| | subfamily A, member 5 | | | involved in olfactory |
| OR10AG1 | olfactory receptor, family | 1.83 | 0.0038 | sensory perception |
| | 10, subfamily AG, member | | | |
| | 1 | | | |
| OR4C11 | olfactory receptor, family 4, | 1.75 | 0.0404 | |
| | subfamily C, member 11 | | | |
| OR1L8 | olfactory receptor, family 1, | 1.7 | 0.0202 | |
| | subfamily L, member 8 | | | |
| OR4C3 | olfactory receptor, family 4, | 1.7 | 0.0311 | |
| | subfamily C, member 3 | | | |
| OR8J1 | olfactory receptor, family 8, | 1.7 | 0.0035 | |
| | subfamily J, member 1 | | | |
| OR2T10 | olfactory receptor, family 2, | 1.62 | 0.0255 | |
| | subfamily T, member 10 | | | |
| OR4A5 | olfactory receptor, family 4, | 1.61 | 0.0156 | |
| | subfamily A, member 5 | | | |
| OR51F2 | olfactory receptor, family | 1.61 | 0.0143 | |
| | 51, subfamily F, member 2 | | | |

### Experimental Example 7

### (Microarray Analysis of Mouse Hippocampus)

The hippocampi of mice subjected to the tail suspension test in Experimental Example 3 were excised, and changes in gene expression were analyzed through microarray analysis.

After the tail suspension test was performed for 7 days, mice from each group, the 1% linalyl acetate administration group, the 10% linalyl acetate administration group, and the control group, were euthanized by cervical dislocation, the whole brain was excised, and the cerebral cortex, olfactory bulb, and hippocampus were excised. Subsequently, RNA was extracted from the hippocampus using ISOGEN (Nippon Gene), and microarray analysis was performed using Clariom S Assay (Applied Biosystems). 20,000 or more genes were analyzed. Data was analyzed using software such as Database for Annotation and Visualization and Integrated Discovery (DAVID).

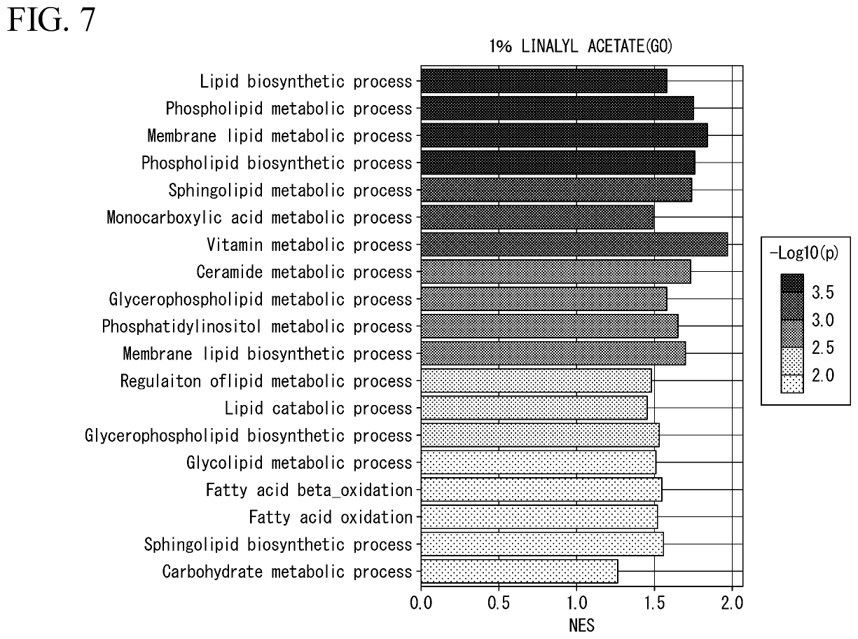

FIG. 7 is a graph showing the results of gene ontology (GO) enrichment analysis of genes with significantly increased expression in the 1% linalyl acetate administration groupmice compared to the control groupmice. From top to bottom, they are shown in order of high statistical significance (−Log 10 p value). The horizontal axis represents normalized enrichment score (NES).

As a result, the significantly enriched processes were related to lipid biosynthetic processes and lipid metabolic processes such as metabolisms of phospholipids, membrane lipids, sphingolipids, ceramides, and glycerophospholipids. In addition, enrichment of vitamin metabolism and carbohydrate metabolism was also observed, indicating that linalyl acetate treatment promoted broad metabolic regulation with a focus on lipid homeostasis.

In GO terms related to lipid biosynthetic processes, lipid biosynthetic process, membrane lipid biosynthetic process, glycerophospholipid biosynthetic process, sphingolipid biosynthetic process, and the like were enriched.

Examples of genes associated with the GO termlipid biosynthetic process include Kl, F5, and Lbp.

Examples of genes associated with the GO term membrane lipid biosynthetic process include Pign, Hacd3, and Hacd4.

Examples of genes associated with the GO termglycerophospholipid biosynthetic process include Abhd4, Inpp5f, and Ptdss1.

Examples of genes associated with the GO termsphingolipid biosynthetic process include Elovl7, Hacd3, and Hacd4.

In GO terms related to lipid metabolism, phospholipid metabolic process, membrane lipid metabolic process, sphingolipid metabolic process, ceramide metabolic process, glycerophospholipid metabolic process, phosphatidylinositol metabolic process, regulation of lipid metabolic process, lipid catabolic process, glycolipid metabolic process, fatty acid beta oxidation, fatty acid oxidation and the like were enriched.

Examples of genes associated with the GO termphospholipid metabolic process include Enpp2, Pla2g7, and Abhd4.

Examples of genes associated with the GO term membrane lipid metabolic process include Enpp2, Gm2a, and Pign.

Examples of genes associated with the GO termsphingolipid metabolic process include Enpp2, Gm2a, and Hacd3.

Examples of genes associated with the GO term ceramide metabolic process include Gm2a, Plpp2, and Plpp3.

Examples of genes associated with the GO termglycerophospholipid metabolic process include Enpp2, Dgkb, and Ocrl.

Examples of genes associated with the GO term phosphatidylinositol metabolic process include Ocrl, Pign, and Inpp5f.

Examples of genes associated with the GO term regulation of lipid metabolic process include Acaa2, Elovl7, and Msmo1.

Examples of genes associated with the GO termlipid catabolic process include Enpp2, Pla2g7, and Pla2g5.

Examples of genes associated with the GO term glycolipid metabolic process include Pign, Gm2a, and Slc30a9.

Examples of genes associated with the GO termfatty acid beta oxidation include Acaa2, Decr1, and Etfdh.

Examples of genes associated with the GO termfatty acid oxidation include Echs1, Eci2, and Crot.

In GO terms related tovitamin metabolism, vitamin metabolic process and the like were enriched.

Examples of genes associated with the GO termvitamin metabolic process include Ttr, Enpp2, and Rbp1.

In GO terms related tocarbohydrate metabolism, monocarboxylic acid metabolic process, carbohydrate metabolic process, and the like were enriched.

Examples of genes associated with the GO termmonocarboxylic acid metabolic process include Cyp39a1, Ucp2, and Fads2.

Examples of genes associated with the GO termcarbohydrate metabolic process include Kl, Ucp2, and Foxo1.

The predominance of lipid-related categories indicated a shift toward enhancement of lipid biosynthesis, remodeling, and energy metabolism, suggesting that administration of linalyl acetate may contribute to maintaining the neuronal membrane integrity and synaptic function in the hippocampus.

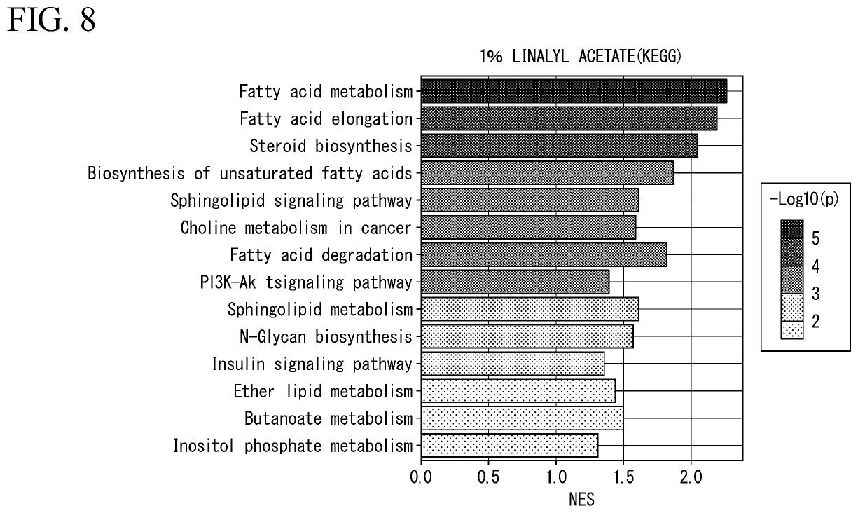

FIG. 8 is a graph showing the results of KEGG pathway analysis of genes with significantly increased expression in the 1% linalyl acetate administration group mice compared to the control group mice. From top to bottom, they are shown in order of high statistical significance (−Log 10 p value). The horizontal axis represents normalized enrichment score (NES).

As a result, the significantly enriched pathways included fatty acid metabolism, fatty acid elongation, steroid biosynthesis, biosynthesis of unsaturated fatty acids, sphingolipid metabolism and signaling pathway. In addition, the enrichment of the PI3K-Akt signaling pathway and insulin signaling pathway was also detected. These signaling pathways played important roles in cell survival, plasticity, and energy regulation.

The above results indicate that, when linalyl acetate was administered, the lipid metabolism network was regulated, the signaling pathways related to cell growth and survival were activated and thus neuroprotective effects in the hippocampus were exhibited.

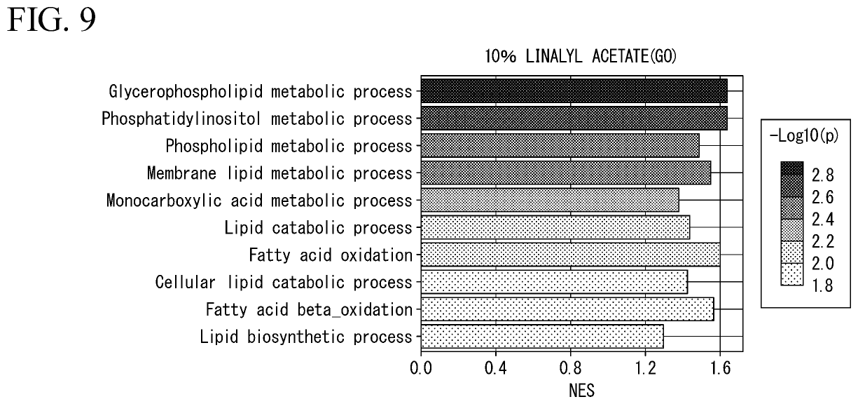

FIG. 9 is a graph showing the results of gene ontology (GO) enrichment analysis of genes with significantly increased expression in the 10% linalyl acetate administration group mice compared to the control group mice. From top to bottom, they are shown in order of high statistical significance (−Log 10 p value). The horizontal axis represents normalized enrichment score (NES).

As a result, the significantly enriched processes were related to glycerophospholipid metabolism, phosphatidylinositol metabolism, phospholipid metabolism, and membrane lipid metabolism, and also included monocarboxylic acid metabolism. In addition, several degradation processes such as lipid catabolic process, fatty acid oxidation process, and cellular lipid degradation process were also enriched.

These results indicate that administration of linalyl acetate not only promoted lipid remodeling and membrane homeostasis, but also enhanced lipid utilization and energy release, thereby regulating hippocampus metabolism more dynamically under stress conditions.

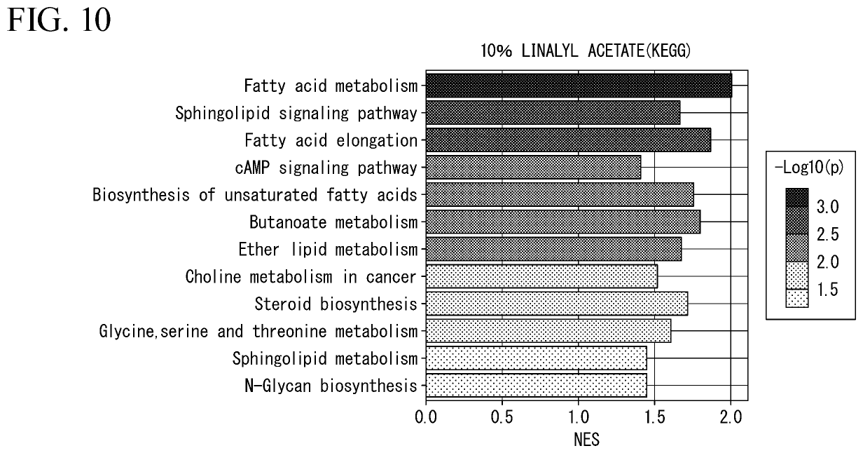

FIG. 10 is a graph showing the results of KEGG pathway analysis of genes with significantly increased expression in the 10% linalyl acetate administration group mice compared to the control group mice. From top to bottom, they are shown in order of high statistical significance (−Log 10 p value). The horizontal axis represents normalized enrichment score (NES).

As a result, the significantly enriched pathways included fatty acid metabolism, fatty acid elongation, biosynthesis of unsaturated fatty acids, sphingolipid signaling, and sphingolipid metabolism. In addition, the enrichment of the cAMP signaling pathway and PI3K-Akt signaling pathway was also observed. These pathways were important for synapse plasticity, neurogenesis, and stress resistance. In addition, pathways such as butanoate metabolism, glycine, serine, and threonine metabolism, and steroid biosynthesis exhibited broad effects on neuro transmitter balance, energy homeostasis, and steroid-mediated signaling.

Based on the above results of this experimental example, it was confirmed that administration of linalyl acetate exhibited a dose-dependent metabolic reprogramming effect in the hippocampus. It is shown that linalyl acetate at a low dose primarily stimulated biosynthesis and protective signaling, and linalyl acetate at a high dose additionally activated degradation and energy production pathways, and regulated a broader range of neurotransmitters and signal networks, for example, the cAMP signaling pathway and amino acid metabolism. These metabolic effects were considered to secure structural resilience and energy balance in the hippocampus under stress-induced conditions and to potentially form the basis for the anti-depressant effects and neuroprotective effects.

According to the present invention, it is possible to provide an agent for regulating neurological function that contains odor molecules as an active component. Since movement of olfactory cilia having olfactory receptors and activation of olfactory ciliary organization improve the functions of olfactory cells, not only curing of olfactory disorders but also the effects of preventing and improving neurodegenerative diseases such as depression and schizophrenia are expected.

While preferred embodiments of the invention have been described and illustrated above, it should be understood that these are exemplary examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A method for treating olfactory disorders, multiple sclerosis, metachromatic leukodystrophy, paralysis, sensory disorders, cognitive dysfunction, Alzheimer's disease, amyotrophic lateral sclerosis, schizophrenia, bipolar disorders, stress, fatigue or anxiety disorders, comprising:
administering an effective amount of linalyl acetate, α-terpineol or 1,8-cineole to a subject in need thereof.

2. The method according to claim 1, wherein the administering promotes the expression of genes involved in olfactory cilia movement or olfactory ciliary organization in nasal epithelial cells.

3. The method according to claim 2, wherein the genes involved in olfactory cilia movement or olfactory ciliary organization include genes associated with the gene ontology terms cilium movement, cilium movement involved in cell motility, cilium or flagellum-dependent cell motility, cilium-dependent cell motility, and cilium organization.

4. The method according to claim 1, wherein the administering promotes the expression of genes involved in lipid biosynthesis, lipid metabolism, vitamin metabolism or carbohydrate metabolism in the hippocampus.

5. The method according to claim 4,
wherein the genes involved in lipid biosynthesis include genes associated with the gene ontology terms lipid biosynthetic process, membrane lipid biosynthetic process, glycerophospholipid biosynthetic process, and sphingolipid biosynthetic process,
the genes involved in lipid metabolism include genes associated with the gene ontology terms phospholipid metabolic process, membrane lipid metabolic process, sphingolipid metabolic process, ceramide metabolic process, glycerophospholipid metabolic process, phosphatidylinositol metabolic process, regulation of lipid metabolic process, lipid catabolic process, glycolipid metabolic process, fatty acid beta oxidation, and fatty acid oxidation,
the genes involved in vitamin metabolism include genes associated with the gene ontology term vitamin metabolic process, and
the genes involved in carbohydrate metabolism include genes associated with the gene ontology terms monocarboxylic acid metabolic process and carbohydrate metabolic process.
